(19) United States
(12) Patent Application Publication
(10) Pub. No.: US 2020/0029634 A1
(43) Pub. Date: Jan. 30, 2020

(54) ARM COVER GLOVE DEVICE FOR PROTECTING THE REMOVAL OF INTRAVENOUS ACCESS DEVICE AND METHOD OF USE THEREOF

(71) Applicants: Billy Lance WILSON, Rockingham, NC (US); Jessica BRIGMAN, Rockingham, NC (US)

(72) Inventors: Billy Lance WILSON, Rockingham, NC (US); Jessica BRIGMAN, Rockingham, NC (US)

(21) Appl. No.: 16/046,895

(22) Filed: Jul. 26, 2018

(51) Int. Cl. A41D 13/08; A41D 19/00; A41D 27/10; A61M 25/02

(52) U.S. Cl. CPC A41D 13/08; A41D 19/0013; A41D 19/0034; A41D 27/10; A61M 2025/0213; A41D 2600/00; A41D 2400/32; A61M 2025/0206; A61M 25/02

(57) ABSTRACT

An arm cover glove device for protecting the removal of an intravenous access device includes a sleeve portion, a glove portion and a securing system. The sleeve portion is configured to go around an arm of a patient. The sleeve portion includes an open top end configured to receive and cover an arm of the patient into a hollow interior of the sleeve portion, and a bottom end. The glove portion is connected to the bottom end of the sleeve portion. The glove portion is configured to receive and cover the hand of the patient. The sleeve portion and the glove portion are configured to cover the intravenous access device while connected to the arm or hand of the patient. The securing system is configured to keep the patient from removing the arm cover glove device.

### FIELD OF THE DISCLOSURE

The present disclosure is directed toward patient care with Intravenous Therapy (“IV”). More specifically, the present disclosure is related to an arm cover glove device for protecting the removal of an intravenous access device and method of use thereof.

### BACKGROUND

Intravenous therapy (“IV”) is a therapy that delivers liquid substances directly into a vein. The intravenous route of administration can be used for injections (with a syringe at higher pressures) or infusions (typically using only the pressure supplied by gravity). Intravenous infusions are commonly referred to as drips. The intravenous route is currently known as the fastest way to deliver medications and fluid replacement throughout the body, because the circulation carries them. Intravenous therapy may be used for fluid replacement (such as correcting dehydration), to correct electrolyte imbalances, to deliver medications, and for blood transfusions. As a result, IVs are very common throughout the medical field in and out of hospitals around the world.

Intravenous systems can be categorized by which type of vein the inserted tube or access device, called the catheter, empties into. A peripheral line is used on peripheral veins (the veins in the arms, hands, legs and feet). This is the most common type of IV therapy used. Central IV lines have their catheters that are advanced through a vein and empty into a large central vein (a vein within the torso), usually the superior vena cava, inferior vena cava or even the right atrium of the heart. Finally, a third type is a midline catheter which is inserted into a peripheral vein and advances through the vein, unlike a peripheral IV line, but falls short of emptying into a central vein.

The instant disclosure may be best suited for use with peripheral line IVs with access devices positioned in the arms and hands. With these types of peripheral line IVs, nurses across the country, in a variety of settings, face some of the same frustrating issues every day. One such issue is the fact that during nurse shifts, some of the patients, especially those that suffer from altered mental status, constantly remove their intravenous access devices. The nurse then must take the time to insert another IV. This issue costs the facility money in materials and in the nurse's time. The nurse also must divert her attention away from other patients to attend to this issue. Also, having to continually stick the patient increases the patients risk of infection. For example, with patients with altered mental status, it is not uncommon to stick a patient more than ten times in one twelve-hour shift.

The instant disclosure is designed to address at least certain aspects of the problems or needs discussed above by providing an arm cover glove device for protecting the removal of an intravenous access device and method of use thereof.

### SUMMARY

Accordingly, in one aspect, the present invention embraces an arm cover glove device. The arm cover glove device may be for protecting the removal of an intravenous access device. The arm cover glove device may generally include a sleeve portion, a glove portion and a securing system. The sleeve portion may be configured to go around an arm of a patient. The sleeve portion may include an open top end configured to receive and cover an arm of the patient into a hollow interior of the sleeve portion, and a bottom end. The glove portion may be connected to the bottom end of the sleeve portion. The glove portion may be configured to receive and cover the hand of the patient. The sleeve portion and the glove portion may be configured to cover the intravenous access device while connected to the arm or hand of the patient. The securing system may be configured to keep the patient from removing the arm cover glove device.

In select embodiments of the arm cover glove device of the instant disclosure for protecting the removal of the intravenous access device, the securing system may include at least one strap to deter removal of the device. The at least one strap may be configured to keep the open top end of the sleeve portion at a top portion of the arm of the patient. In select embodiments, the securing system may include a first strap and a second strap. The first strap may be configured to connect from a first connection point on a front side of the open top end of the sleeve portion around a neck of the patient to a second connection point on a back side of the open top end of the sleeve portion. The second strap may be configured to connect from the first connection point around a torso of the patient to the second connection point, where the second strap goes under an opposite arm of the patient. In select embodiments, the first strap and the second strap may be adjustable in length to accommodate various size patients. In other select embodiments, the first strap may include a neck pad configured to protect the neck of the patient from the first strap, and/or the second strap may include a torso pad configured to protect the torso of the patient from the second strap. Yet, in other select embodiments of the securing system, a third strap may be included. The third strap may be connected between the first strap and the second strap approximately below the neck or an opposite shoulder of the patient. Wherein, the third strap may be configured to prevent the patient from raising the first strap over a head of the patient.

One feature of the instant arm cover glove device of the instant disclosure for protecting the removal of the intravenous access device may be that the first strap and the second strap may be securely connected to the front side of the open top end of the sleeve portion at the first connection point, and the first strap and the second strap may be removably connected to the back side of the open top end of the sleeve portion at the second connection point. With this configuration, the patient may not access the removable connections of the first strap and the second strap for removing the arm cover glove device.

Another feature of the instant arm cover glove device of the instant disclosure for protecting the removal of the intravenous access device may be that the sleeve portion and the glove portion may be integral.

In select embodiments of the instant arm cover glove device of the instant disclosure for protecting the removal of the intravenous access device, the open top end of the sleeve portion may include an elastic member. The elastic member of the open top end of the sleeve portion may be configured to aid in holding the open top end around a top portion of the arm of the patient.

In select embodiments of the instant arm cover glove device of the instant disclosure for protecting the removal of the intravenous access device, the sleeve portion and the glove portion may be made from a flexible material.

Another feature of the instant arm cover glove device of the instant disclosure for protecting the removal of the intravenous access device may be that the glove portion can include open finger portions. The open finger portions may be configured to allow at least finger tips of the hand of the patient to be exposed. In select embodiments, the open finger portions may be configured to allow whole fingers of the patient to be exposed, where the glove portion covers a palm and a back side of the hand of the patient.

In another aspect, the instant disclosure embraces an IV system. The IV system of the instant disclosure may generally include a peripheral line with the intravenous access device and the arm cover glove device of the instant disclosure. The arm cover glove device may be included in any of the various embodiments shown and/or described herein. The intravenous access device of the peripheral line may be connected to an arm or a hand of a patient. The arm cover glove device may be configured for protecting removal of the intravenous access device from the arm or hand of the patient. Wherein, the intravenous access device of the peripheral line is covered by the arm cover glove device.

One feature of the instant IV system according to the instant disclosure may be that at least one tube from the peripheral line runs from the arm or hand inside the arm cover glove device to the outside of the arm cover glove device via the open top end.

In select embodiments of the IV system of the instant disclosure, the at least one tube may be connected to an infusion bag or a syringe.

In another aspect, the instant disclosure embraces a method of preventing the removal of an intravenous access device. The method of preventing the removal of an intravenous access device of the instant disclosure may generally include the step of providing the arm cover glove device for protecting the removal of an intravenous access device in any of the various embodiments shown and/or described herein. The method of preventing the removal of an intravenous access device of the instant disclosure may include the steps of: connecting the peripheral line to the arm or the hand of the patient; inserting the arm of the patient into the open top end of the sleeve portion and positioning the hand inside the glove portion; and securing the securing system around the patient.

In select embodiments of the method of preventing IV removal of the instant disclosure, wherein the patient suffers from altered mental status: the step of connecting the peripheral line to the arm or the hand of the patient may include connecting the peripheral line to the arm or the hand of an altered mental status patient; the step of inserting the arm of the patient into the open top end of the sleeve portion and positioning the hand inside the glove portion may include inserting the arm of the altered mental status patient into the open top end of the sleeve portion and positioning the hand inside the glove portion; and the step of securing the securing system around the patient may include securing the securing system around the altered mental status patient.

The foregoing illustrative summary, as well as other exemplary objectives and/or advantages of the invention, and the manner in which the same are accomplished, are further explained within the following detailed description and its accompanying drawings.

### BRIEF DESCRIPTION OF THE DRAWINGS

The present intravenous protector will be better understood by reading the Detailed Description with reference to the accompanying drawings, which are not necessarily drawn to scale, and in which like reference numerals denote similar structure and refer to like elements throughout, and in which:

FIG. 1 is an environmental prospective view of an intravenous therapy (“IV”) setup in a standard hospital bed according to the prior art;

FIG. 2 is an environmental prospective view of select embodiments of the IV system with the arm cover glove device for protecting the removal of an intravenous access device according to the instant disclosure being used in a standard hospital bed;

FIG. 3 is a front view of select embodiments of the arm cover glove device for protecting the removal of an intravenous access device according to the instant disclosure;

FIG. 4 is a zoomed-in front view of the arm cover glove device for protecting the removal of an intravenous access device from FIG. 3;

FIG. 5 is a rear view of select embodiments of the arm cover glove device for protecting the removal of an intravenous access device according to the instant disclosure;

FIG. 6 is a zoomed-in rear view of the arm cover glove device for protecting the removal of an intravenous access device from FIG. 5; and

FIG. 7 is a flow diagram of the method of protecting the removal of an intravenous access device according to select embodiments of the instant disclosure.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

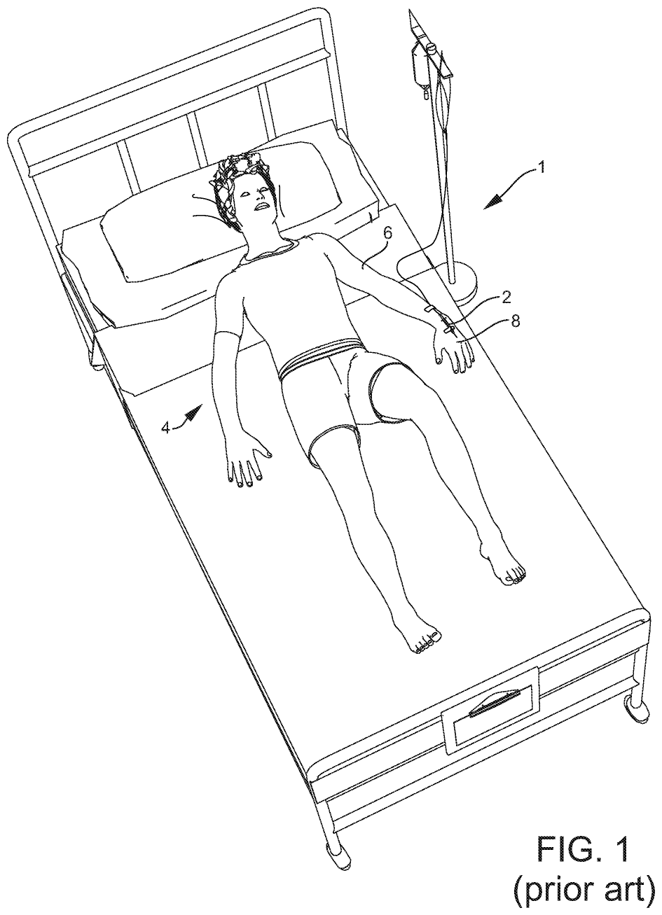

Referring first to FIG. 1, an environmental prospective view of an intravenous therapy (“IV”) setup 1 in a standard hospital bed is shown according to the prior art. As shown, the commonly used IV setup 1 of the prior art includes a peripheral line IV with intravenous access device 2 positioned in hand 8 on arm 6 of patient 4. There is no covering or protection for preventing the removal of intravenous access device 2 from hand 8. As such, it is common that patient 4 may accidentally or even purposely remove intravenous access device 2 from hand 8. As such, the instant disclosure recognizes that nurses across the country, in a variety of settings, face some of the same frustrating issues every day, where during nurse shifts, some of the patients, especially those that suffer from altered mental status, constantly remove their intravenous access device, like intravenous access device 2 of IV setup 1, shown in FIG. 1. The nurse then must take the time to insert another IV or intravenous access device. This issue costs the facility money in materials and in the nurse's time. The nurse also must divert her attention away from other patients to attend to this issue. Also, having to continually stick the patient increases the patients risk of infection. For example, with patients with altered mental status, it is not uncommon to stick a patient more than ten times in one twelve-hour shift.

The instant disclosure may thus be designed to address at least certain aspects of the problems or needs discussed above regarding FIG. 1 of the prior art, by providing arm cover glove device 10 for protecting the removal of intravenous access device 12 and method 200 of use thereof.

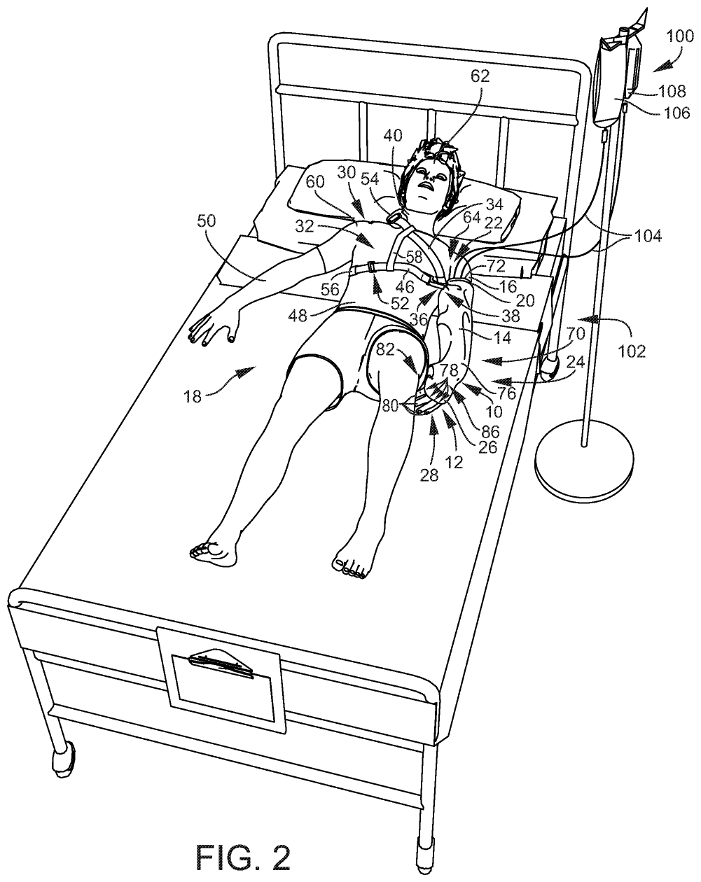

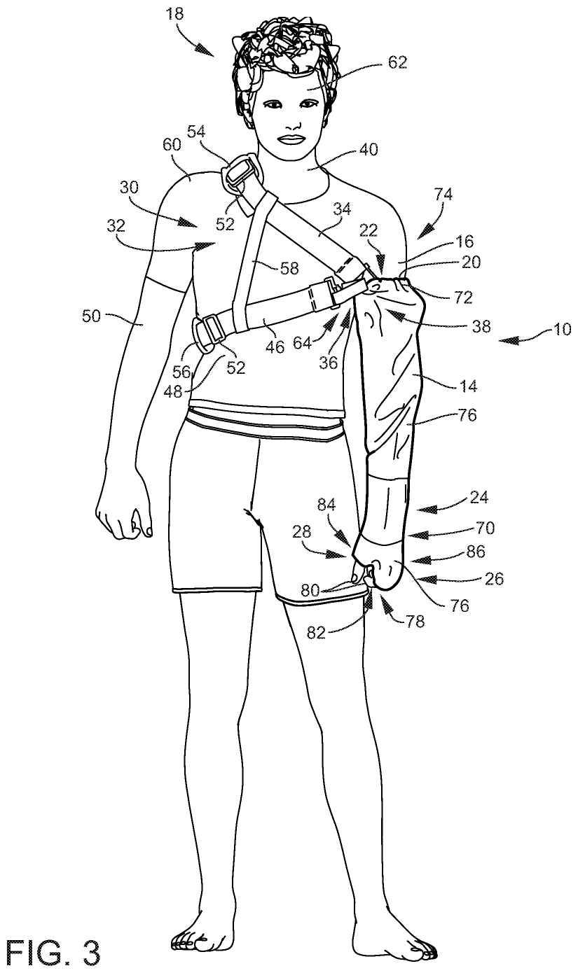

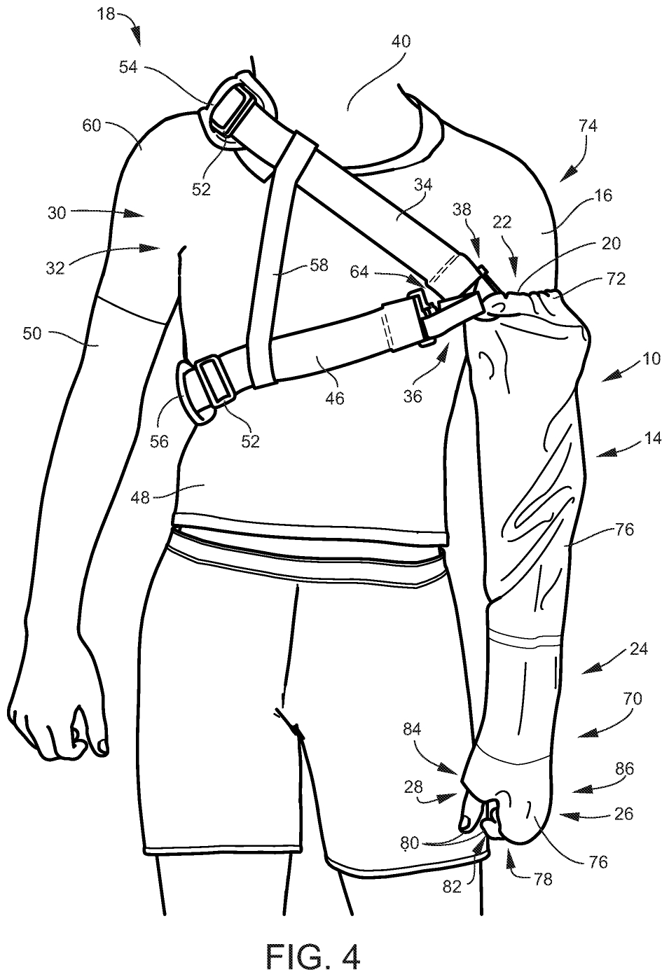

Referring now to the instant disclosure, as shown in FIGS. 2-7, in describing the exemplary embodiments of the present disclosure, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples and are merely examples among other possible examples.

Referring now specifically to FIGS. 2-6, in a possibly preferred embodiment, the present disclosure overcomes the above-mentioned disadvantages and meets the recognized need for such an apparatus or method by providing of arm cover glove device 10. Arm cover glove device 10 may be for protecting or preventing removal of intravenous access device 12. Arm cover glove device may generally include sleeve portion 14, glove portion 26, and securing system 30. These components and their features and functions will be described in more detail below.

Sleeve portion 14 may be included with arm cover glove device 10. Sleeve portion 14 may be for covering arm 16 of patient 18. In select embodiments, sleeve portion 14 may cover the full arm 16 or a majority of arm 16 of patient 18, as shown in FIGS. 2-6. As such, sleeve portion 14 may be configured to go around arm 16 of patient 18. Sleeve portion 14 may include open top end 20. Open top end 20 may be configured to receive and cover arm 16 of patient 18 into hollow interior 22 of sleeve portion 14. Sleeve portion 14 may also include bottom end 24. Bottom end 24 may connect to glove portion 26. As shown in FIGS. 2-6, in select embodiments of arm cover glove device 10 for protecting the removal of intravenous access device 12, open top end 20 of sleeve portion 14 may include elastic member 72. Elastic member 72 around the periphery of open top end 20 of sleeve portion 14 may be configured to aid in holding open top end 20 around top portion 74 of arm 16 of patient 18. Elastic member 72 may be sewn into the periphery of open top end 20, similar to the elastic waste of shorts or undergarments. Along with aiding in holding open top end 20 on top portion 74 of arm 16, elastic member 72 may allow for tubes 104 from intravenous access device 12 to protrude from open top end 20 of sleeve portion 14 of arm cover glove device 10 for connecting infusion bag 106 or syringe 108 with intravenous access device 12 inside arm cover glove device 10.

Glove portion 26 may be included with arm cover glove device 10. Glove portion 26 may be for covering hand 28 of arm 16 of patient 18. Glover potion 26 may be connected to bottom end 24 of sleeve portion 14. As such, when patient 18 inserts hand 28 into open top end 20 and inside hollow interior 22 of sleeve portion 14, hand 28 may be slid down inside hollow interior 22 into glove portion 26. As such, glove portion 26 may be configured to receive and cover hand 28 of patient 18. As shown in FIGS. 2-6, another feature of arm cover glove device 10 for protecting the removal of intravenous access device 12 may be that glove portion 26 can include open finger portions 78. Open finger portions 78 may be configured to allow at least finger tips 80 of hand 28 of patient 18 to be exposed. This exposure of at least finger tips 80 through open finger portions 78 of glove portion 26 may provide for more natural use of hand 28 for patient 18 as well as added comfort to patient 18. In select embodiments, open finger portions 78 may be configured to allow whole fingers 82 of patient 18 to be exposed, where glove portion 26 merely covers palm 84 and back side 86 of hand 28 of patient 18. By providing exposure of whole fingers 82 of patient 18 through open finger portions 78 of glove portion 26, patient 18 may be provided with the most natural use of hand 28 as well as the most added comfort to patient 18 while still covering and protecting the removal of intravenous access device 12 under arm cover glove device 10. While health care providers will be responsible for removing arm cover glove device 10 and accessing the IV access site with intravenous access device 12, open finger portions 78 may also allow providers to assess for swelling, cyanosis, drawing, the like, etc.

As shown in FIGS. 2, in combination, sleeve portion 14 and glove portion 26 may be configured to cover intravenous access device 12 while connected to arm 16 or hand 28 of patient 18.

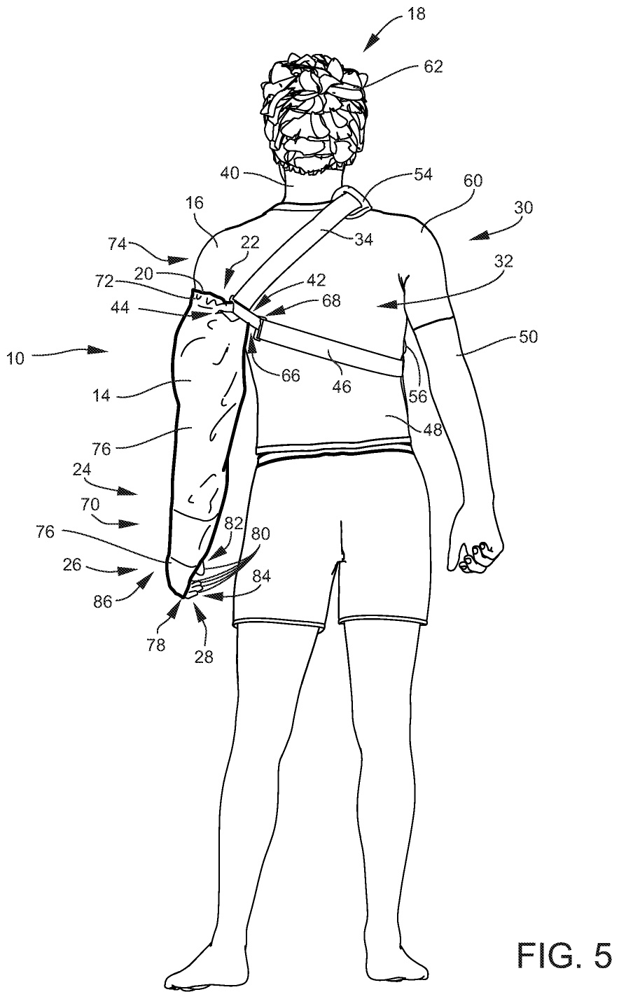

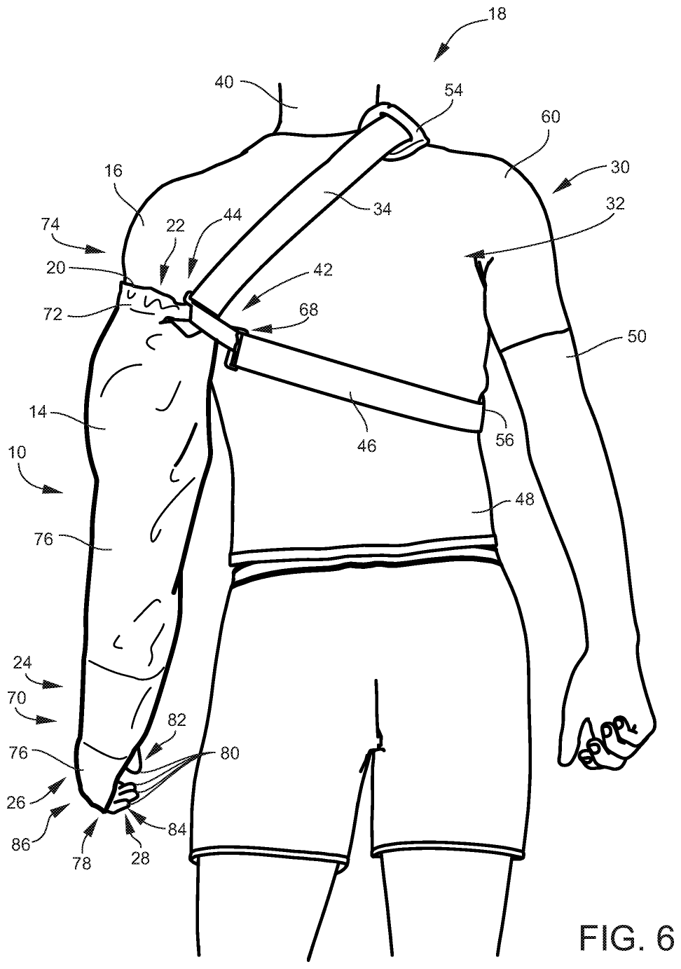

Securing system 30 may be included with arm cover glove device 10. Securing system 30 may be included for keeping arm cover glove device 10 on patient 18. As such, securing system 30 may be configured to keep patient 18 from removing arm cover glove device 10. Securing system 30 may include any device, devices, mechanisms, the like, etc. configured for keeping arm cover glove device 10 on patient 18, including any device, devices, mechanisms, the like, etc. configured to keep patient 18 from removing arm cover glove device 10. In select embodiments of arm cover glove device 10, securing system 30 may include at least one strap 32. At least one strap 32 may be configured to keep open top end 20 of sleeve portion 14 at top portion 74 of arm 16 of patient 18. In select embodiments, securing system 30 may include first strap 34 and/or second strap 46. As shown in FIGS. 2-6, in select embodiments, securing system 30 may include first strap 34 and second strap 46. First strap 34 may be configured to connect from first connection point 36 on front side 38 (see FIGS. 2-4) of open top end 20 of sleeve portion 14 around neck 40 of patient 18 to second connection point 42 on back side 44 (see FIGS. 5-6) of open top end 20 of sleeve portion 14. Second strap 46 may be configured to connect from first connection point 36 on front side 38 (see FIGS. 2-4) of open top end 20 of sleeve portion 14 around torso 48 of patient 18 to second connection point 42 on back side 44 (see FIGS. 5-6) of open top end 20 of sleeve portion 14. Within this configuration, second strap 46 may go under opposite arm 50 of patient 18. In select optional embodiments, first strap 34 and/or second strap 46 may be adjustable in length to accommodate various size patients. As such, as shown in the FIGS. 2-4, first strap 34 and/or second strap 46 may include adjustable portions 52 for adjusting their lengths to accommodate various size patients. In other select optional embodiments, first strap 34 may include neck pad 54 configured to protect neck 40 of patient 18 from first strap 34, and/or second strap 46 may include torso pad 56 configured to protect torso 48 of patient 18 from second strap 46. Neck pad 54 and/or torso pad 56 may be any devices or materials configured for protecting neck 40 and torso 48 from rubbing, scratching, pressure relieve, the like, etc., from first strap 34 and/or second strap 46. Yet, in other select embodiments of securing system 30, third strap 58 may also be included (see FIGS. 2-4). Third strap 58 may be connected between first strap 34 and second strap 46. In select embodiments, third strap 58 may be connected between first strap 34 and second strap 46 approximately below neck 40 or an opposite shoulder 60 of patient 18, so somewhere therebetween. Wherein, third strap 58 may be configured to prevent patient 18 from raising first strap 34 over head 62 of patient 18 for unleashing securing system 30 and removing arm cover glove device 10.

Referring specifically to FIGS. 2-4, one feature of securing system 30 of arm cover glove device 10 for protecting the removal of intravenous access device 12 may be that first strap 34 and second strap 46 may be securely connected to front side 38 of open top end 20 of sleeve portion 14 at first connection point 36. This secure connection 64 on front side 38 of arm cover glove device 10 may prevent patient 18 from removing first strap 34 and/or second strap 46, thereby, freeing patient 18 to remove arm cover glove device 10. This secure connection 64 on front side 38 of arm cover glove device 10 may be any secured connection, or connection that would be impossible or at least difficult for patient 18 to remove. As examples, and clearly not limited thereto, secure connection 64 on front side 38 of arm cover glove device 10 may be a sewn connection, a melted connection, a glued connection, a riveted connection, the like, etc.

Referring now specifically to FIGS. 5-6, another feature of arm cover glove device 10 for protecting the removal of intravenous access device 12 may be that first strap 34 and second strap 46 may be removably connected to back side 44 of open top end 20 of sleeve portion 14 at second connection point 42. This removable connection 66 on back side 44 of arm cover glove device 10 may allow securing system 30 of arm cover glove device 10 to be secured on patient 18 with the help of a nurse or the like. However, this removable connection 66 on back side 44 of arm cover glove device 10 may prevent patient 18 from removing first strap 34 and/or second strap 46 by having removable connection 66 in non-accessible location 68 to patient 18. This removable connection 66 on back side 44 of arm cover glove device 10 may be any removable connection. As examples, and clearly not limited thereto, removable connection 66 on back side 44 of arm cover glove device 10 may be include any ties, snaps, hook and loop type fasteners, closures, buckles, clasps, clips, catches, fastenings, carabiners, the like, etc.

As shown in FIGS. 2-6, another feature of the instant arm cover glove device 10 for protecting the removal of intravenous access device 12 may be that sleeve portion 14 and glove portion 26 may be integral. In other words, sleeve portion 14 and glove portion 26 may be formed from the same material with integral connection 70. In other select embodiments, sleeve portion 14 and glove portion 26 may be formed from separate materials and connected together. In other select embodiments of arm cover glove device 10, sleeve portion 14 and glove portion 26 may be made from flexible material 76. Flexible material 76 may be any common flexible glove or sleeve type material whether currently known, or later discovered, including, but not limited to, any sterile hospital materials, like latex, rubber, the like, etc.

Referring now specifically to FIG. 2, in another aspect, the instant disclosure embraces IV system 100. IV system 100 of the instant disclosure may generally include peripheral line 102 with intravenous access device 12 and arm cover glove device 10. Arm cover glove device 10 of IV system 100 may be in any of the various embodiments shown and/or described herein. Intravenous access device 12 of peripheral line 102 may be connected to arm 16 or hand 28 of patient 18 similar to the prior art of FIG. 1. Arm cover glove device 10 may be configured and used for protecting removal of intravenous access device 12 from arm 16 or hand 28 of patient 18. Wherein, intravenous access device 12 of peripheral line 102 can then be covered and protected by arm cover glove device 10, as shown in FIG. 2. One feature of IV system 100 may be that at least one tube 104 from peripheral line 102 may run from arm 16 or hand 28 inside arm cover glove device 10 to the outside of arm cover glove device 10 via open top end 20 of sleeve portion 14. As examples, and clearly not limited thereto, as shown in FIG. 2, in select embodiments of IV system 100, the at least one tube 104 may be connected to infusion bag 106 for various gravity infusion purposes (like hydration) or to syringe 108 for forced introduction of fluids, or pushed introduction of fluids into intravenous access device 12.

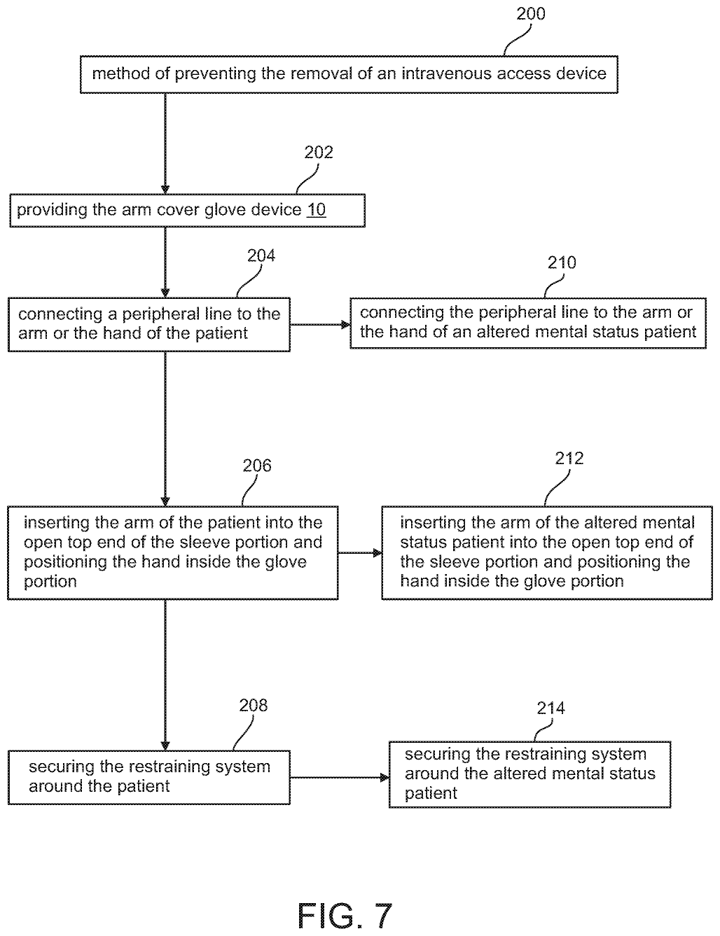

Referring now specifically to FIG. 7, in another aspect, the instant disclosure embraces method 200 of preventing the removal of intravenous access device 12. Method 200 of preventing the removal of intravenous access device 12 may generally include step 202 of providing arm cover glove device 10 for protecting the removal of intravenous access device 12 in any of the various embodiments shown and/or described herein. As a result, method 200 of preventing the removal of intravenous access device 12 may further include the steps of: step 204 of connecting peripheral line 102 to arm 16 or hand 28 of patient 18; step 206 of inserting arm 16 of patient 18 into open top end 20 of sleeve portion 14 and positioning hand 28 inside glove portion 26; and step 208 of securing, applying or connecting securing system 30 around patient 18.

In select embodiments of method 200 of preventing IV removal, where patient 18 suffers from altered mental status, step 204 of connecting peripheral line 102 to arm 16 or hand 28 of patient 18 may include step 210 of connecting peripheral line 102 to arm 16 or hand 28 of an altered mental status patient. Likewise, step 206 of inserting arm 16 of patient 18 into open top end 20 of sleeve portion 14 and positioning hand 28 inside glove portion 26 may include step 212 of inserting arm 16 of the altered mental status patient into open top end 20 of sleeve portion 14 and positioning hand 28 inside glove portion 26; and step 208 of securing, applying or connecting securing system 30 around patient 18 may include step 214 of securing, applying or connecting securing system 30 around the altered mental status patient.

In sum, the present disclosure is directed to a full-length arm sleeve device 10 that serve the purpose of protecting intravenous access device 12 from accidental or purposeful removal or tampering. Arm cover glove device 10 can also be used to protect arm 16 of patient 18 for any other reason deemed necessary or important. Arm cover glove device 10 may be targeted for, but not limited to, clients that suffer from altered mental status. Arm cover glove device may be designed so that the patients, themselves, will not be able to remove it. However, arm cover glove device 10 can be quickly removed by staff or the patient's family in case of an emergency.

Sleeve portion 14 will extend from top portion 74 of arm 16 of patient 18, down the forearm, over the biggest part of hand 28, and reach between the fingers, leaving all digits 82 exposed for full use. The goal may be to ensure that the IV is protected from tampering no matter where it is placed on the arm and/or that the arm is protected for any other purposes. First strap 34 may be attached to front side 38 of open top end 20 of sleeve portion 14, where it reaches around neck 40 of patient 18 and attaches to back side 44 of sleeve portion 14 via removable connection 66 with adjustable device 52 used to ensure the best fit. Also, adjustable second strap 46 will extend from front side 38 of open top end 20 of sleeve portion 14 on arm 16, over the torso 48 of patient 18, under opposite arm 50, and secured to back side 44 of open top end 20 of sleeve portion 14 of arm cover glove device 10.

As a result, arm cover glove device 10 with a full-length or almost full-length arm sleeve portion 14 may serve the purpose of protecting intravenous access device 12 from accidental or purposeful removal or tampering. Arm cover glove device 10 can also be used to protect arm 16 of patient 18 for any other reason deemed necessary or important. Arm cover glove device 10 may be targeted for, but not limited to, clients that suffer from altered mental status. Arm cover glove device 10 may be designed so that the patients, themselves, will not be able to remove it; however, arm cover glove device 10 can be quickly removed by staff or the patient's family in case of an emergency.

In the specification and/or figures, typical embodiments of the disclosure have been disclosed. The present disclosure is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

The foregoing description and drawings comprise illustrative embodiments. Having thus described exemplary embodiments, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments will come to mind to one skilled in the art to which this disclosure pertains having the benefit of the teachings presented in the foregoing descriptions and the associated drawings. Although specific terms may be employed herein, they are used in a generic and descriptive sense only and not for purposes of limitation. Accordingly, the present disclosure is not limited to the specific embodiments illustrated herein but is limited only by the following claims.

1. An arm cover glove device for protecting removal of an intravenous access device comprising:
a sleeve portion configured to go around an arm of a patient, said sleeve portion including:
an open top end configured to receive and cover the arm of the patient into a hollow interior of the sleeve portion; and
a bottom end;
a glove portion connected to the bottom end of the sleeve portion, said glove portion is configured to receive and cover a hand of the patient;
said sleeve portion and said glove portion are configured to cover the intravenous access device while connected to the arm or the hand of the patient; and
a securing system configured to keep the patient from removing the arm cover glove device.

2. The arm cover glove device of claim 1, wherein the securing system including at least one strap configured to keep the open top end of the sleeve portion at a top portion of the arm of the patient.

3. The arm cover glove device of claim 2, wherein the securing system including:
a first strap configured to connect from a first connection point on a front side of the open top end of the sleeve portion around a neck of the patient to a second connection point on a back side of the open top end of the sleeve portion; and
a second strap configured to connect from the first connection point around a torso of the patient to the second connection point, where the second strap is positioned under an opposite arm of the patient.

4. The arm cover glove device of claim 3, wherein the first strap and the second strap are adjustable in length to accommodate various size patients.

5. The arm cover glove device of claim 3, wherein:
the first strap including a neck pad configured to protect the neck of the patient from the first strap; and
the second strap including a torso pad configured to protect the torso of the patient from the second strap.

6. The arm cover glove device of claim 3 further comprising a third strap, the third strap is connected between the first strap and the second strap approximately below the neck or an opposite shoulder of the patient, wherein the third strap is configured to prevent the patient from raising the first strap over a head of the patient.

7. The arm cover glove device of claim 3, wherein:
the first strap and the second strap are securely connected to the front side of the open top end of the sleeve portion at the first connection point; and
the first strap and the second strap are removably connected to the back side of the open top end of the sleeve portion at the second connection point;
wherein, the patient cannot access the second connection point with the first strap and the second strap removably connected for removing the arm cover glove device.

8. The arm cover glove device of claim 1, wherein the sleeve portion and the glove portion are integral.

9. The arm cover glove device of claim 1, wherein the open top end of the sleeve portion including an elastic member configured to aid in holding the open top end around a top portion of the arm of the patient.

10. The arm cover glove device of claim 1, wherein the sleeve portion and the glove portion are made from a flexible material.

11. The arm cover glove device of claim 1, wherein the glove portion including open finger portions configured to allow at least finger tips of the hand of the patient to be exposed.

12. The arm cover glove device of claim 11, wherein the open finger portions are configured to allow whole fingers of the patient to be exposed, where the glove portion is covering a palm and a back side of the hand of the patient.

13. An IV system comprising:
a peripheral line with an intravenous access device connected to an arm or a hand of a patient; and
an arm cover glove device configured for protecting removal of the intravenous access device from the arm or the hand of the patient, said arm cover glove device comprising:
a sleeve portion configured to go around an arm of the patient, said sleeve portion including:
an open top end configured to receive and cover an arm of the patient into a hollow interior of the sleeve portion; and
a bottom end;
a glove portion connected to the bottom end of the sleeve portion, said glove portion is configured to receive and cover the hand of the patient;
said sleeve portion and said glove portion are configured to cover the intravenous access device while connected to the arm or the hand of the patient; and
a securing system configured to keep the patient from removing the arm cover glove device;
wherein, the intravenous access device of the peripheral line is covered by the arm cover glove device.

14. The IV system of claim 13, wherein at least one tube from the peripheral line is connected from the arm or the hand inside the arm cover glove device to the outside of the arm cover glove device via the open top end.

15. The IV system of claim 14, wherein the at least one tube is connected to an infusion bag or a syringe.

16. The IV system of claim 13, wherein the securing system including:
a first strap configured to connect from a first connection point on a front side of the open top end of the sleeve portion around a neck of the patient to a second connection point on a back side of the open top end of the sleeve portion, the first strap including a neck pad configured to protect the neck of the patient from the first strap;
a second strap configured to connect from the first connection point around a torso of the patient to the second connection point, where the second strap is positioned under an opposite arm of the patient, the second strap including a torso pad configured to protect the torso of the patient from the second strap; and
a third strap, the third strap is connected between the first strap and the second strap approximately below the neck or an opposite shoulder of the patient, wherein the third strap is configured to prevent the patient from raising the first strap over a head of the patient;
wherein the first strap and the second strap are adjustable in length to accommodate various size patients.

17. The IV system of claim 16, wherein:
the first strap and the second strap are securely connected to the front side of the open top end of the sleeve portion at the first connection point; and
the first strap and the second strap are removably connected to the back side of the open top end of the sleeve portion at the second connection point;
wherein, the patient cannot access the second connection point with the first strap and the second strap removably connected for removing the arm cover glove device.

18. The IV system of claim 13, wherein:
the sleeve portion and the glove portion are integral;
the open top end of the sleeve portion including an elastic member configured to aid in holding the open top end around a top portion of the arm of the patient;
the sleeve portion and the glove portion are made from a flexible material;
the glove portion including open finger portions configured to allow at least finger tips of the hand of the patient to be exposed, wherein the open finger portions are configured to allow whole fingers of the patient to be exposed, where the glove portion merely covers a palm and back side of the hand of the patient.

19. A method of preventing the removal of an intravenous access device comprising:
providing an arm cover glove device for protecting removal of the intravenous access device comprising:
a sleeve portion configured to go around an arm of a patient, said sleeve portion including:
an open top end configured to receive and cover the arm of the patient into a hollow interior of the sleeve portion; and
a bottom end;
a glove portion connected to the bottom end of the sleeve portion, said glove portion is configured to receive and cover a hand of the patient;
said sleeve portion and said glove portion are configured to cover the intravenous access device while connected to the arm or the hand of the patient; and
a securing system configured to keep the patient from removing the arm cover glove device;
connecting a peripheral line to the arm or the hand of the patient;
inserting the arm of the patient into the open top end of the sleeve portion and positioning the hand inside the glove portion; and
securing the securing system around the patient.

20. The method of preventing IV removal of claim 19, wherein the patient is an altered mental status patient, whereby:
connecting the peripheral line to the arm or the hand of the patient including connecting the peripheral line to the arm or the hand of the altered mental status patient;
inserting the arm of the patient into the open top end of the sleeve portion and positioning the hand inside the glove portion including inserting the arm of the altered mental status patient into the open top end of the sleeve portion and positioning the hand inside the glove portion; and
securing the securing system around the patient including securing the securing system around the altered mental status patient.
